(19) United States
(12) Patent Application Publication
(10) Pub. No.: US 2006/0293641 A1
(43) Pub. Date: Dec. 28, 2006

(54) Housing body for deglutition of solid molding for oral ingestion, container for the housing body, and deglutition method

(76) Inventors: Fuyuhiko Nishijima, Tokyo (JP); Saichi Ono, Tokyo (JP); Tadahiko Chiba, Tokyo (JP); Yukio Iwase, Tokyo (JP); Yoshiaki Osaka, Tokyo (JP); Koichi Niimura, Tokyo (JP)

Correspondence Address: REED SMITH LLP, 3110 FAIRVIEW PARK DRIVE, FALLS CHURCH, VA, 22042, US

(21) Appl. No.: 10/556,592

(22) PCT Filed: May 14, 2004
(86) PCT No.: PCT/JP04/06544

§ 371 Date: Nov. 14, 2005

(30) Foreign Application Priority Data
May 14, 2003 (JP) 2003-135841

(51) Int. Cl. A61M 31/00

(52) U.S. Cl. 604/500

(57) ABSTRACT

A housing body (10) for deglutition of a shaped solid (5) for oral ingestion which is stored therein, comprising a generally tubular water-resistant envelope (1); an opening-side end part (A) at one end of the envelope, having an unsealable structure part (2), and capable of forming an opening part (22) by removing the unsealable structure part; a shielding-side end part (B) at the other end of the envelope, having a means for preventing the shaped solid for oral ingestion from release, the means allowing liquid to pass therethrough but not allowing the shaped solid for oral ingestion to pass therethrough; and a storage chamber (6) storing the shaped solid for oral ingestion between the opening-side end part and the shielding-side end part is disclosed. A method for swallowing a shaped solid for oral ingestion, wherein an unsealable structure part installed at an opening-side end part of the housing body is removed; the shielding-side end part of the housing body is immersed into liquid in a vessel while the shaped solid for oral ingestion is stored in the storage chamber in the tubular envelope; and an opening part formed at the opening-side end part is held between the lips so that the shaped solid for oral ingestion is sucked in together with the liquid in the vessel is also disclosed.

### TECHNICAL FIELD

The present invention relate to a housing body for deglutition of a shaped solid for oral ingestion, a container for use in the housing body, and a swallowing method. The housing body of the present invention contains the shaped solid for oral ingestion, such as powders (including fine granules), granules, capsules, tablets, or pills in a drinking straw-like cylindrical envelope thereof in a storage state. Thus, as when drinking water or the like through a drinking straw, the shaped solid can be swallowed with water in a glass after immersing the tip of the cylindrical envelope into the water in the glass. For example, a relatively large capsule or tablet can be swallowed very easily.

### BACKGROUND ART

When a capsule or tablet is ingested, in general, the capsule or tablet is first held in the mouth, water is then taken in from a glass or cup, and the capsule or tablet is swallowed together with the water without chewing. Nevertheless, many patients feel repulsion and pain when taking a relatively large medical drug, such as a capsule or tablet, and many patients cannot take a capsule or tablet without taking in a large quantity of water.

Further, for patients suffering from a renal or cardiac disease, an amount of water that may be taken in is limited. Such patients are requested to minimize the amount of water taken in when ingesting a capsule or tablet. Therefore, patients who essentially need a large quantity of water when taking a capsule or tablet feel an intense pain upon doing so. Furthermore, it may be difficult for an aged person or a child to swallow a capsule or tablet, and other formulations are highly desired.

As mentioned below, the inventors of the present invention have found that a drinking straw can eliminate such pain. Thus, prior art utilizing a drinking straw will first be described.

For example, Japanese Unexamined Patent Publication (Kokai) No. 2002-104541 (patent reference 1) discloses a manner wherein powders, granules, syrup or the like stored in an elongated sachet having a water-resistance and strength are introduced into a vessel such as a cup, and then the emptied sachet is used as a stirrer and a drinking straw. In this prior art, water readily-soluble or readily-dispersible powders or granules are encapsulated in the straw, and immediately before use, is dropped down into water in a glass and dissolved or dispersed, and the resulting drinkable liquid is sucked in through the straw.

Japanese Translation Publication (Kohyo) No. 2001-502173 (patent reference 2) discloses an embodiment similar to that disclosed in patent reference 1. Further, patent reference 2 also discloses an embodiment wherein beverage is sucked through a straw containing a flavouring agent coated on a surface of an inner wall of the straw, and an embodiment wherein beverage is sucked through a straw containing a flavouring agent between filters installed at the both ends thereof, while the flavouring agent is being dissolved.

Patent reference 1 and patent reference 2, however, cover only a beverage prepared by dissolving or dispersing a water readily-soluble or readily-dispersible powder or granule in water. In the case of the dispersed beverage, granules having a very small particle size are taken into consideration, but fine granules or granules having a rather larger size are not taken into consideration.

A powdery or granular medical drug for oral administration is generally supplied in the form of a dividedly wrapping heat-sealed film, or an elongated sachet. For example, Japanese Translation Publication (Kohyo) No. 2002-528483 (patent reference 3) discloses a straw-like container having therein granular drugs, and Japanese Patent No. 2607422 (patent reference 4) discloses a separately packaged container of an adsorbent for oral administration. In these cases, however, powdery or granular medical drugs are generally taken in the powdery or granular form. Persons, i.e., patients, who must take the powdery or granular medical drugs hold them in their mouth, and then swallow them down with water supplemented from a glass or the like. When a large amount of the powdery or granular drugs is to be taken, they sometimes are inserted into mouth after being wrapped with a wafer sheet.

When a large number of capsules or tablets are taken as a single unit dose, the total single unit is often divided several times, each taken with water. Thus, a larger amount of water becomes necessary. Under the circumstances, Japanese Unexamined Patent Publication (Kokai) No. 11-124342 (patent reference 5) discloses an oral jelly for aiding a swallowing of medical drugs.

Nevertheless, there were disadvantages in the manner of the conventional oral administration. For example, powdery or granular medical drugs are liable to be spilled when inserting from a package to the mouth, or transferring from a package to a wafer sheet. When the wafer sheet or the oral jelly is used, procedures prior to actual use are cumbersome. Further, the powdery or granular medical drugs may choke a person, i.e., a patient, taking the same, when inserted into the mouth, and be spilled out therefrom.

[patent reference 1]

Japanese Unexamined Patent Publication (Kokai) No. 2002-104541
   [patent reference 2]
Japanese Translation Publication (Kohyo) No. 2001-502173
   [patent reference 3]
Japanese Translation Publication (Kohyo) No. 2002-528483
   [patent reference 4]
Japanese Patent No. 2607422
   [patent reference 5]
Japanese Unexamined Patent Publication (Kokai) No. 11-124342

### DISCLOSURE OF THE INVENTION

Problems to be Solved by the Invention

As above, for a water readily-soluble or readily-dispersible powder or granule, various techniques for supplying a drinkable form after conversion to a drinkable liquid have been proposed. For a water practically insoluble or insoluble powder or granule, however, a means for a convenient ingestion thereof has not been proposed, even with water. Further, for a capsule or tablet to be taken in a larger amount as a single unit dose, a means for a convenient ingestion thereof has not been proposed, even with water.

Accordingly, the object of the present invention is to provide a means for easily taking a water practically insoluble or insoluble powder (including fine granule), granule, capsule, tablet, or pill together with water.

Means for Solving the Problems

The present invention relates to a housing body for deglutition of a shaped solid for oral ingestion which is stored therein, comprising
(1) a tubular water-resistant envelope;
(2) an opening-side end part at one end of the envelope, having an unsealable structure part, and capable of forming an opening part by removing the unsealable structure part;
(3) a shielding-side end part at the other end of the envelope, having a means for preventing the shaped solid for oral ingestion from release, the means allowing liquid to pass therethrough but not allowing the shaped solid for oral ingestion to pass therethrough; and
(4) a storage chamber storing the shaped solid for oral ingestion between the opening-side end part and the shielding-side end part.

The present invention also relates to a method for swallowing a shaped solid for oral ingestion, characterized in that
(1) an unsealable structure part installed at an opening-side end part of the above housing body, and an unsealable structure part optionally installed at a shielding-side end part thereof are removed,
(2) the shielding-side end part of the housing body is immersed in a liquid in a vessel while the shaped solid for oral ingestion is stored in the storage chamber in the tubular envelope, and
(3) an opening part formed by removing the unsealable structure part installed at the opening-side end part is held between the lips so that the shaped solid for oral ingestion is sucked in or can be sucked in together with the liquid in the vessel.

Further, the present invention relates to a container for use in a housing body for deglutition of a shaped solid for oral ingestion, comprising
(1) a tubular water-resistant envelope;
(2) an opening-side end part at one end of the envelope, having an unsealable structure part, and capable of forming an opening part by removing the unsealable structure part;
(3) a shielding-side end part at the other end of the envelope, having a means for preventing the shaped solid for oral ingestion from release, the means allowing liquid to pass therethrough but not allowing the shaped solid for oral ingestion to pass therethrough; and
(4) a storage chamber capable of storing the shaped solid for oral ingestion between the opening-side end part and the shielding-side end part.

Furthermore, the present invention relates to a method for swallowing a shaped solid for oral ingestion, characterized in that
(1) an unsealable structure part installed at an opening-side end part of the above container is removed,
(2) the shaped solid for oral ingestion is introduced into the storing chamber of the tubular envelope from an opening part formed by removing the unsealable structure part installed at the opening-side end part,
(3) the shielding-side end part is immersed in a liquid in a vessel while the shaped solid for oral ingestion is stored in the storage chamber, and
(4) an opening part at the opening-side end part is held between the lips so that the shaped solid for oral ingestion is sucked in or can be sucked in together with liquid in the vessel.

Still further, the present invention relates to a container for use in a housing body for deglutition of a shaped solid for oral ingestion, comprising
(1) a tubular water-resistant envelope;
(2) an opening-side end part at one end of the envelope,
(3) a shielding-side end part at the other end of the envelope, having a means for preventing the shaped solid for oral ingestion from release, the means allowing liquid to pass therethrough but not allowing the shaped solid for oral ingestion to pass therethrough; and
(4) a storage chamber capable of storing the shaped solid for oral ingestion between the opening-side end part and the shielding-side end part.

Still further, the present invention relates to a method for swallowing a shaped solid for oral ingestion, characterized in that
(1) the shaped solid for oral ingestion is introduced into the storing chamber of the tubular envelope from the opening part of the opening-side end part of the container according to claim 16,
(2) the shielding-side end part is immersed in a liquid in a vessel while the shaped solid for oral ingestion is stored in the storage chamber, and
(3) the opening part at the opening-side end part is held between the lips so that the shaped solid for oral ingestion is sucked in or can be sucked in together with liquid in the vessel.

### EFFECTS OF THE INVENTION

According to the present invention, a means for easily ingesting a water practically insoluble or insoluble powder (including fine granule), granule, capsule, tablet, or pill together with water is provided. An aged person or a child can also ingest the same without a change in the formulations.

### BEST MODE FOR CARRYING OUT THE INVENTION

The housing body of the present invention contains therein the shaped solid for oral ingestion, such as capsules or tablets in a straw-like cylindrical envelope in a stored state. When the shaped solid for oral ingestion is to be taken, the unsealable structure part of the opening-side end part is removed to form an opening part, and the tip of the shielding-side end part is dipped into water in a cup. Then, when water or the like is drunk through a straw, the shaped solid can be drunk or swallowed together with water. Therefore, a large number of water practically insoluble or insoluble powders including fine granules can be easily swallowed, and capsules, tablets or pills having a relatively larger size can be easily drunk or swallowed together with a small amount of water.

In the conventional manner, capsules or tablets are first inserted into a cavitas oris and then water is added from a glass or the like to thereby enable them to be swallowed. In this case, when the capsules or tablets are inserted into a cavitas oris, the throat acts to block the conveying of the capsules or tablet to the esophagus. Further, the capsules or tablets are randomly placed in different directions in the cavitas oris, and thus are liable to choke the throat when swallowed together with water. In the case of the water practically insoluble or insoluble powders (including fine granules) and granules, these spread throughout the mouth and produce a problem of an uncomfortable feel remaining.

In the present invention, on the contrary, a person taking drugs will drink water through a straw as though no shaped solid exists in the cavitas oris. Thus, the throat acts to positively convey water to the esophagus. At the same time, the water practically insoluble or insoluble powders (including fine granules), granules, capsules, tablets, pills, etc. are guided toward the esophagus. When the capsules, tablets or the like are arranged in the straw in such a direction that they are easily swallowed, they pass through the cavitas oris and the throat along the water stream while maintaining their arranged directions, and thus, they can be very smoothly swallowed, and an amount of water taken in becomes remarkably small, as demonstrated in working Examples as mentioned below. Further, as the capsules, tablets or the like can be taken without directly observing same, there is little feeling of repulsion.

Specific embodiments of the housing body of the present invention will be described hereinafter, referring to affixed drawings.

FIG. 1 is a partially sectional view of one embodiment of the housing body 10 of the present invention. The housing body 10 is mainly composed of a tubular envelope 1. One end is an opening-side end part A and the other end is a shielding-side end part B. A shape of the tubular envelope 1 is not limited, but is for example, a tube having a polygonal or star-shaped cross section. Preferably the tube is cylindrical or elliptical. The shaped solids 5 for oral ingestion are stored therein.

The opening-side end part A has an unsealable structure part 2. An opening part 22 can be formed by removing the unsealable structure part 2. The unsealable structure part 2 preferably has a removing-aiding means such as a groove 21. The opening part 22 can be formed by cutting the tip off along the groove 21. Therefore, the opening-side end part A is closed before the groove 21 is cut off along the groove 21. The groove 21 may be set continuously on, discontinuously (i.e., in a perforated form) on, or in a part of the circumference of the side wall of the tubular envelope 1. Depending on the properties (such as hygroscopic property) of the shaped solid 5 for oral ingestion stored in the storage chamber 6, the opening-side end part A is preferably airtight when closed. If an airtight closure is not necessary, through-holes can be set, instead of the groove 21, discontinuously (i.e., in a perforated form) on the circumference of the side wall of the tubular envelope 1, or a detachable cap may be used.

The other end opposite to the opening-side end part A in the tubular envelope 1 is the shielding-side end part B. The shielding-side end part B contains a means for preventing the shaped solid for oral ingestion from release, that is, allowing liquid to pass therethrough but not allowing the shaped solid for oral ingestion to pass therethrough. The means for preventing the shaped solid for oral ingestion from release may be, for example, a net 32 covering a whole cross-sectional area of the tubular envelope 1, as shown in FIG. 1. The housing body according to the present invention containing such a net will be hereinafter referred to as a net-type housing body.

In the net-type housing body 10 of the present invention as shown in FIG. 1, not only the entire opening part of the shielding-side end part B of the tubular envelope 1, but also the outside edge of the wall of the tubular envelope 1 can be covered with the net 32, and the net 32 can be fixed at the outside edge of the wall of the tubular envelope 1 with a locking ring 8 from the outside of the net 32. The net 32 has a sieve which allows liquid to pass therethrough but does not allow the shaped solid 5 for oral ingestion to pass therethrough, and thus the shaped solid 5 for oral ingestion stored in the tubular envelope 1 can be held therein without passage of the shaped solid 5 for oral ingestion.

The net-type housing body 10 of the present invention as shown in FIG. 1 may contain, for example, a detachable protective cap 46 which may crown the outside of the locking ring 8, as shown in FIG. 2. Further, instead of the fixing the net 32 with the locking ring 8 as shown in FIG. 1, the net 32 may be fixed as shown in FIG. 3 with the locking cap 81 at the outside edge of the tubular envelope 1 from the outside of the tubular envelope 1, and an unsealable structure part 4 may be installed at the tip of the locking cap 81. An opening part 42 can be formed by removing the unsealable structure part 4. The unsealable structure part 4 preferably has a removing-aiding means such as a groove 41. The groove 41 may be set continuously on, discontinuously (i.e., in a perforated form) on, or in a part of the circumference of the side wall of the locking cap 81. Instead of the groove 41, through-holes can be set, discontinuously (i.e., in a perforated form) on the circumference of the side wall of the locking cap 81.

When the net-type housing body 10 of the present invention as shown in FIG. 1 is used, the unsealable structure part 2 is first opened. When the shielding-side end part B contains the protective cap 46 as shown in FIG. 2, the protective cap 46 is removed. Further, if the shielding-side end part B contains the locking cap 81 having the unsealable structure part 4 at the edge thereof, as shown in FIG. 3, the unsealable structure part 4 is opened. When the opening-side end part A is opened, the opening part 22 is formed at the edge. Thus, if the opening part 22 is slanted downward in to a gravitational direction, the shaped solid 5 for oral ingestion stored in the tubular envelope 1 will fall out. Thus, the shielding-side end part B must be slanted downward.

After the unsealable structure part 2 is opened, or optionally the protective cap 46 of the shielding-side end part B is taken off, and the unsealable structure part 4 is opened, as shown in FIG. 4, the unsealed housing body 11 is immersed in a liquid (such as water) 62 of a vessel 61 such as a glass from the shielding-side end part B, while the shielding-side end part B is slanted downward and the shaped solid 5 for oral ingestion is maintained as stored in the storage chamber 6 of the unsealed housing body 11.

Subsequently, as shown in FIG. 4, the unsealable structure part 2 is opened to form the opening part 22, and the resulting opening part 22 is held between the lips of a person taking the drugs. When the person uses the unsealed housing body 11 of the present invention as a drinking straw to suck in water 62 in the cup 61, water 62 is drawn as shown by the arrow X from the shielding-side end part B, and sucked upward as shown by the arrow Y inside the tubular envelope 1. At the same time, the shaped solid 5 for oral ingestion is also sucked in together with the water upwardly in the direction of the arrow Y, through the lips and the cavitas oris, and into the throat of the person at a single gulp, and is swallowed.

FIG. 5 is a partial sectional view illustrating another embodiment of the shielding-side end part B in the net-type housing body 10 of the present invention. In the embodiment shown in FIG. 5, the net 33 is installed at the edge of the shielding-side end part B in the net-type housing body 10. It can be prepared, for example, by covering the entire opening part of the shielding-side end part B in the tubular envelope 1 and the tip of the outside of the tubular envelope 1 with the net 33 as shown in FIG. 1, then adhering the net 33 to the edge of the opening part of the shielding-side end part B in the tubular envelope 1 by means of heat-sealing or the like, and thereafter, cutting off an outside portion of the net from the heat-sealed part. In this embodiment, it is not necessary to use the locking ring 8 as shown in FIG. 1.

FIG. 6 is a partial sectional view illustrating still another embodiment of the shielding-side end part B in the net-type housing body 10 of the present invention. The embodiment shown in FIG. 6 has the net 31 placed on the inner wall of the tubular envelope 1 in the net-type housing body 10. It can be prepared, for example, by joining a cylindrical ring to the net-covered edge as shown in FIG. 5 from the outside thereof. In this embodiment, it is also not necessary to use the locking ring 8 as shown in FIG. 1.

As shown in FIG. 7, the net-type housing body 10 as shown in FIG. 5 may contain the detachable protective cap 46 at the shielding-side end part B. Further, as shown in FIG. 8, the net-type housing body 10 as shown in FIG. 6 may contain the unsealable structure part 4 at the shielding-side end part B. The opening part 42 may be formed by removing the unsealable structure part 4. The unsealable structure part 4 preferably has a removing-aiding means such as a groove 41. The groove 41 may be set continuously on, discontinuously (i.e., in a perforated form) or in a part. Instead of the groove 41, through-holes can be set, discontinuously (i.e., in a perforated form).

The shielding-side end part B of the housing body 10 of the present invention may contain a narrow passage portion composed of a single or plural protrusions projecting towards an inner space of the tubular envelope 1 as a means for preventing the shaped solid for oral ingestion from release. The housing body according to the present invention containing such a narrow passage portion will be hereinafter referred to as a narrow passage-type housing body.

FIG. 9 (perspective partial view) and FIG. 10 (sectional partial view) illustrate an embodiment of the shielding-side end part B in the narrow passage-type housing body 10. In the embodiment shown in FIGS. 9 and 10, the opening part 42 is divided into two narrow passages 42a, 42b by joining the central part 79 of the opening part 42 of the shielding-side end part B, for example, by means of heat-sealing. When the narrow passage-type housing body 10 is used as shown in FIG. 4, the narrow passages 42a, 42b allow liquid 62 to pass therethrough, but do not allow the shaped solids 5 for oral ingestion to pass therethrough. Thus, before use, it can prevent the shaped solids 5 for oral ingestion from passing therethrough, to store them within the storage chamber 6 of the narrow passage-type housing body 10. The part which can be heat-sealed is not limited to a portion as shown in FIGS. 9 and 10, but two or more parts may be heat-sealed. A method for forming the above shape is not limited to the heat-sealing method. For example, such a shape can be formed when the straw is extruded.

Another embodiment of the shielding-side end part B in the narrow passage-type housing body 10 is illustrated in FIG. 11 (partial sectional view) and FIG. 12 (sectional view). In the embodiment shown in FIGS. 11 and 12, the circumference of the opening part 42 of the shielding-side end part B is compressed, for example, by thermal deformation with a heated iron tong toward the center of the tubular envelope 1 from four outside points so that the inside diameter of the opening part 42 is reduced. Thus, a narrow opening 42c allowing liquid to pass therethrough, but not allowing the shaped solid 5 for oral ingestion to pass therethrough can be formed. The narrow opening 42c may be shaped when the tubular envelope 1 is molded.

FIG. 13 is a partial sectional view illustrating still another embodiment of the shielding-side end part B in the narrow passage-type housing body 10 of the present invention. In the embodiment shown in FIG. 13, the circular circumference of the opening part 42 of the shielding-side end part B is joined at four points 43a, 43b, 43c, 43d, for example, by a thermal deformation or a heat-sealing method, so that the inside diameter of the opening part 42 is reduced. Thus, a narrow opening 42c allowing liquid to pass therethrough, but not allowing the shaped solid 5 for oral ingestion to pass therethrough can be formed. In the embodiment, the narrow opening 42c may be formed by heat-sealing 1 to 3 points or 5 or more points of the circular circumference of the opening part 42.

FIG. 14 is a partial sectional view illustrating still another embodiment of the shielding-side end part B in the narrow passage-type housing body 10 of the present invention. In the embodiment shown in FIG. 14, the narrow passage 42d may be formed by pressing a heated pin-like iron tong toward the center of the tubular envelope 1 from the outside of the tubular envelope 1 at one or more portions thereof in the slightly axially inner area of the opening part 42 in the shielding-side end part B to form protrusions 71a, 71b on the inner wall of the tubular envelope 1. The number of the protrusions is not limited. Alternatively, the narrow passage may be formed by applying a heated iron ring to the outside of the tubular envelope 1, and rotating it thereon to gradually press it toward the center of the tubular envelope 1 and thereby form a ring protrusion on the inner wall of the tubular envelope 1.

FIG. 15 is a partial sectional view illustrating still another embodiment of the shielding-side end part B in the narrow passage-type housing body 10 of the present invention. In the embodiment shown in FIG. 15, the narrow passage 42d may be formed in the slightly axially inner area of the opening part 42 in the shielding-side end part B by forming protrusions 71c, 71d on the inner wall of the tubular envelope 1. The number of the protrusions is not limited. Alternatively, the narrow passage may be formed by forming a ring protrusion on the inner wall of the tubular envelope 1. The protrusion on the inner wall of the tubular envelope 1 may be formed when the tubular envelope 1 is molded, or alternatively by first forming the tubular envelope 1 having protrusions at the edge and then joining a ring tubular envelope 1 to the outside thereof.

FIG. 16 is a partial sectional view illustrating still another embodiment of the shielding-side end part B in the narrow passage-type housing body 10 of the present invention. When the shielding-side end part B of the embodiment shown in FIG. 16 is produced, for example, plural strips 74a, 74b, 74c, 74d, 74e, 74f, 74g, 74h are formed as shown in FIG. 17 (partial sectional view) by cutting the shielding-side end part B of the tubular envelope 1 axially from the edge along the cutting lines 73. Then, the strips 74a, 74b, 74c, 74d, 74e, 74f, 74g, 74h are divided into two groups, i.e., a group of strips to be bent toward the inside, and a group of strips not to be bent but maintained as they are. For example, as shown in FIG. 16, the narrow opening 42e can be formed by dividing the strips alternately to the strips 74a, 74c, 74e, 74g not to be bent, and the strips 74b, 74d, 74f, 74h to be bent inward. The number of the total strips and the number of strips to be bent are not limited.

FIG. 18 is a partial sectional view illustrating still another embodiment of the shielding-side end part B in the narrow passage-type housing body 10 of the present invention. When the shielding-side end part B of the embodiment shown in FIG. 18 is produced, for example, as shown in FIG. 19 (partial sectional view), the shielding-side end part B in the tubular envelope 1 is completely closed by forming a heat-sealed portion 75 at the edge of the shielding-side end part B, and a removable portion 76 is installed in a part of the closed edge. The removable portion 76 preferably has a removing-aiding means such as a groove 76a. The groove 76a may be set continuously or discontinuously (i.e., in a perforated form). Instead of the groove 76a, through-holes can be set, discontinuously, i.e., in a perforated form. The narrow opening 42f can be formed at a position where the removable portion 76 is removed. The number of removable portions set at the edge of the shielding-side end part B is not limited. For example, an embodiment of the shielding-side end part B having two removable portions 76, 77 at the heat-sealed part 75 is shown in FIG. 20, a partial sectional view. Immediately before using the narrow passage-type housing body 10 of the embodiment shown in FIGS. 18 and 19, the removable portion 76 can be removed to form the narrow opening 42f at the same time as the opening-side end part A at the other end is unsealed, to thereby provide the unsealed housing body 11. Alternatively, it can be supplied to a person to take drugs in the form of the narrow passage-type housing body 10 having the narrow opening 42f which has been formed by removing the removable portion 76 in advance. The person can then convert it to the unsealed housing body 11 by unsealing the opening-side end part A at the other end before use.

A simple narrow passage portion can be formed at an intermediate part of the tubular water-resistant envelope. For example, the Harrow passage 42d composed of the protrusions 71a, 71b shown in FIG. 14 or the narrow passage 42d composed of the protrusions 71c, 71d shown in FIG. 15 may be formed at the position axially toward the opening-side end part A far from the opening part 42 of the shielding-side end part B, that is, at the intermediate position of the tubular water-resistant envelope. Further, the intermediate portion of the tubular water-resistant envelope made of a deformable synthetic resin material may be squashed with the fingers, or deformed by folding, to form the narrow passage portion. The narrow passage portion may be formed by heating, curling and deforming the intermediate portion of the tubular water-resistant envelope made of a deformable thermoplastic resin material with the heat from lighter flame or the like.

The housing body of the present invention 10, in the case of either the net-type housing body or the narrow passage-type housing body, may contain the backflow preventing means at the outside of the net 32 or the narrow passage portion placed in the shielding-side end part B. FIG. 21 is a partial sectional view illustrating a backflow preventing cap 9 placed at an outermost edge of the tubular envelope 1 in the net-type housing body 10 as shown in FIG. 5, as the backflow preventing means. The backflow preventing cap 9 is composed of a conical jacket 91, a ring joined part 92, and a ball 90 stored therein. An opening part 93 is formed at the top of the conical jacket 91, and the cap can be fixed to the outside of the tubular envelope 1 at the ring joined part 92.

When the net-type housing body 10 having the backflow preventing cap 9 is used as shown in FIG. 4, after unsealing the opening-side end part A to convert it to the unsealed housing body 11, water 62 sucked from the cup 61 through the opening part 93 formed at the top of the conical jacket 91 draws up the ball 90, and thus, water 62 is drawn through the net 33 as shown by the arrow X from the shielding-side end part B, and sucked upward as shown by the arrow Y inside the tubular envelope 1. When the person taking the drug ceases the sucking, the sucking of the water 62 ceases, and the ball 90 falls in the direction of gravitational force and closes the opening part 93 formed at the top of the conical jacket 91. Therefore, water 62 remains within the conical jacket 91, and a backflow to the vessel 61 can be prevented.

A shape of the tubular envelope is not limited in the present invention, so long as the object of the present invention is achieved. An essential sectional shape is similar to a straw used for drinking, and preferably is cylindrical or elliptically cylindrical. A method for forming such a shape is not limited. A flow passage is not limited to the linear from one end to the other, but may be spiral or curved from one end to the other end. Further, the tubular water-resistant envelope may be a linear envelope having a spiral or curved portion, or a portion transformable to a curved form, as a part thereof.

FIG. 22 illustrates the unsealed housing body 11A containing a curved portion C. The unsealed housing body 11A corresponds to an embodiment wherein the unsealed housing body 11 as shown in FIG. 4 contains the curved portion C at a portion near to the opening-side end part A in the unsealed housing body 11. If the curved portion C is contained at the portion above the upper edge of the vessel 61 when the unsealed housing body 11A is inserted into the vessel 61 such as a cup, as shown in FIG. 22, the housing body for deglutition of the present invention may be used, for example, while lying on a bed or the like. The parts similar to those shown in FIG. 4 are identified by the same numbers in FIG. 22.

FIG. 23 illustrates the unsealed housing body 11B containing a portion D (for example, an accordion-folding portion) transformable to a curved form. The unsealed housing body 11B corresponds to an embodiment wherein the accordion-folding portion D is placed instead of the curved portion C in the unsealed housing body 11A shown in FIG. 22 at the same position thereof. It can be also used in the application in the same way as the unsealed housing body 11A shown in FIG. 22, by bending the accordion-folding portion D as shown in FIG. 23.

In the unsealed housing body 11B containing the accordion-folding portion D, the accordion-folding portion D might obstruct a smooth passage of the shaped solid 5 for oral ingestion, depending on the shape of the shaped solid 5 for oral ingestion. For example, when the shaped solids 5 for oral ingestion are large solids, such as tablets or capsules, some solids might not pass through the accordion-folding portion D. When the shaped solids 5 for oral ingestion are powders (including fine granules) or granules, some of them might be trapped by the accordion-folding portion D and remain thereat. To avoid such a non-passing or remaining, it is preferable to store in advance the shaped solid 5 for oral ingestion at a position between the accordion-folding portion D and the opening-side end part A, as shown in FIG. 23. It is more preferable to store the same shortly before use. Particularly, in the case of the large shaped solid such as a tablet or capsule, the accordion-folding portion D serves as the means for preventing the shaped solid for oral ingestion from release, and thus, it becomes unnecessary to install the net 32 or the locking ring 8 in the embodiment as shown in FIG. 23. In this case, the area from the accordion-folding portion D to the opening part for drawing water from the cup corresponds to the shielding-side end part B. The parts similar to those shown in FIG. 4 are identified by the same numbers in FIG. 23.

As mentioned, in the embodiment containing the accordion-folding portion in the tubular water-resistant envelope, the accordion-folding portion can be used as the means for preventing the shaped solid for oral ingestion from release, for a large shaped solid such as a tablet or a capsule. In this case, the accordion-folding portion can be placed at any position in the tubular water-resistant envelope. When used, a longer side having a longer distance between the end of the tubular water-resistant envelope and the accordion-folding portion may be dipped into water in a vessel such as a cup, or a shorter side having a shorter distance between the end of the tubular water-resistant envelope and the accordion-folding portion may be dipped into water in a vessel such as a cup.

In the unsealed housing body 11B shown in FIG. 23, that is, the housing body storing the shaped solid 5 for oral ingestion between the accordion-folding portion D and the opening-side end part A, the shaped solid 5 for oral ingestion must be stored in such a manner that the shaped solid 5 for oral ingestion is not conveyed when a person taking drugs bends the accordion-folding portion D before sucking, but is smoothly conveyed by the sucking of the person. Therefore, it is preferable to wrap the shaped solid 5 for oral ingestion with a water-soluble sheet material 51 such as a wafer, as shown in FIG. 24, i.e., a schematic partial sectional view. Before sucking by the person taking drugs, the conveying is prevented by a friction of the water-soluble sheet material 51 such as a wafer. When water is sucked by the person, the water-soluble sheet material 51 such as a wafer is dissolved and the shaped solid is conveyed together with water. When plural large capsules or tablets having an external diameter almost identical to an inner diameter of the storage chamber 6 are linearly stored in the storage chamber 6, it is sufficient to wrap the first and the last large capsules or tablets with the water-soluble sheet material 51 such as a wafer.

Even if the opening-side end part A in the housing body containing the accordion-folding portion is closed, for example, with the unsealable structure part 2 as in the embodiment shown in FIG. 1, the shaped solid 5 for oral ingestion may fall out when the unsealable structure part 2 is removed to form the opening part 22. Typical means for avoiding this are shown in FIGS. 25 to 29.

FIG. 25 (schematic partial sectional view) illustrates water-soluble protrusions 23 installed on the inner wall of the opening part 22 at the opening-side end part A. The water-soluble protrusions 23 are dissolved when brought into contact with water, and thus, oral ingestion is not interrupted.

FIG. 26 (elevational view) and FIG. 27 (schematic partial sectional view) illustrate an extruding valve 24 made of an elastic material such as a rubber. The extruding valve 24 shown in FIGS. 26 and 27 is composed of a ring support 83 connecting to the inner wall of the storage chamber 6, and eight V-shaped folding strips 84 projecting from the ring support 83 to the center thereof. There are radial gaps between the eight V-shaped folding strips 84. The V-shaped folding strips 84 can maintain their planar shape as shown in FIG. 27(a) when stress is not applied. However, when a person taking the drugs sucks water so that a stress is applied to the center by the shaped solid 5 for oral ingestion, as shown in FIG. 27(b), the eight V-shaped folding strips 84 are bent and the radial gaps 85 are widened. Thus, the shaped solid 5 for oral ingestion can be discharged to the outside from the opening part 22.

FIG. 28 (partial perspective view) and FIG. 29 (schematic partial sectional view) illustrate a sliding openable/closable cap 25 made of a flexible material. FIGS. 28(a) and 29(a) show a closing state of the sliding openable/closable cap 25, whereas FIGS. 28(b) and 29(b) show an opening state. As shown in FIGS. 28(a) and 29(a), the openable/closable cap 25 in the closing state is composed of a cylindrical support 87 at one end, and a conical openable/closable part 88 extending from the cylindrical support 87 to the other end. The conical openable/closable part 88 is composed of plural flexible trapezoidal strips 88a which are divided by plural cutting lines 89. When the openable/closable part 88 is slid in a direction of the arrow S shown in FIGS. 28(a) and 29(a), each flexible trapezoidal strip 88a is outwardly widened by the tubular envelope 1 positioned at the inside thereof, whereby the whole is transformed to a cylindrical form, i.e., the open state, as shown in FIGS. 28(b) and 29(b). Thus, the shaped solid 5 for oral ingestion can be discharged from the open part 22 to the outside. When the openable/closable cap 25 in the open state is slid in a direction opposite to the arrow S, the openable/closable cap 25 is returned to the closed state.

The portion of the shielding-side end part in the housing body of the present invention may be relatively extended. The unsealed housing body 11 of such an embodiment is illustrated in FIG. 30. That is, FIG. 30 shows, as in FIG. 4, the state wherein, after the unsealable structure part of the opening-side end part A is opened, the shielding-side end part B is dipped into a liquid (such as water) 62 in a vessel 61 such as a cup while the shielding-side end part B is slanted downward and the shaped solid 5 for oral ingestion is maintained as stored in the storage chamber 6 of the unsealed housing body 11. The net 31 as shown in FIG. 6 is contained as the means for preventing the shaped solid for oral ingestion from release. As the means for preventing the shaped solid for oral ingestion from release, for example, the narrow passage 42d composed of the protrusions 71a, 71b as shown in FIG. 14, or the narrow passage 42d composed of the protrusions 71c, 71d as shown in FIG. 15 may be used. In these cases, as shown in FIG. 30, it is preferable to place the net or the narrow passage above the liquid level whereby the storing chamber for the capsule or the like is positioned so that the capsule or the like is not brought into contact with water, and thus the surface thereof is not swollen. The parts similar to those shown in FIG. 4 are identified by the same numbers in FIG. 30.

A size, such as a length or an inner diameter, of the tubular envelope in the present invention is not limited, so long as the purpose of the present invention can be achieved. The length when liquid is sucked, that is, a length from the opening part formed after opening the unsealable structure part to the net, has a distance such that the shaped solid for oral ingestion can be sucked together with the liquid (such as water) in a vessel (such as a cup) by holding the opening part between the lips while the net is dipped in the liquid in the vessel. An essential size is same as that of a conventional straw used for drinking. In a preferable embodiment of the housing body of the present invention, a single unit dose of the shaped solid for oral ingestion is stored. In this case, the housing body preferably has a size capable of storing the single unit dose. The single unit dose may be composed of plural tablets or capsules, or of one tablet or capsule. The size of the housing body of the present invention is not limited, but for example, the length of the unsealed housing body is about 50 to 300 mm. The cross-sectional inner diameter is, for example, about 3 to 30 mm, preferably 5 to 15 mm. Further, the thickness of the tubular envelope 1 is, for example, about 0.1 to 5 mm.

The housing body of the present invention is intended not only for packaging, but also for sucking a liquid (in particular water) as a drinking straw while being dipped into the liquid. Therefore, it is made from a material having a strength and resistance to water. When the shaped solid for oral ingestion has hygroscopicity, and an airtight package is required, it is preferably made from an airtight material. Further, it is preferable to overwrap the housing body with a high barrier packaging material or the like, or put the housing body in an airtight bottle. Furthermore, the housing body is used for sucking as a drinking straw, and thus is preferably atoxic.

As a material for the housing body, for example, polyolefin resin, such as polypropylene resin, polyethylene resin, polyester resin, acrylic resin, polyvinyl chloride resin, or polyvinylidene chloride resin, waterproof paper, glass, pottery, or metal may be used. Depending on a method for producing the same, an appropriate sealant layer may be applied.

When the shaped solid for oral ingestion stored in the storage chamber of the housing body has an appearance physiologically unacceptable for oral administration, an opaque housing body may be used to minimize any discomfort of a person taking the shaped solid. On the contrary, when such a discomfort of a person taking the shaped solid can be reduced if the shaped solid can be observed from the outside, a transparent housing body may be used.

Any means may be used as a means for opening the unsealable structure part. For example, it is preferable to use a means such as the groove which does not require a tool, but can be easily opened by hand. Particularly, a method for a manually tearable cover, or opening a lid such as a cap is preferable. The net is not limited, so long as it has a sieve that does not allow the shaped solid for oral ingestion stored in the storage chamber of the unsealed housing body to pass therethrough, but allows liquid to pass therethrough when the liquid is sucked up.

The shaped solid for oral ingestion is stored in the storage chamber of the housing body. The term “shaped solid for oral ingestion” as used herein means, for example, medicines, supplments, foods, beverages or the like, as will be mentioned, and as a shape, for example, of the medicine, powders (including fine granules), granules, capsules, tablets, or pills which are described in the 14th Revised Japanese Pharmacopoeia, and is not limited so long as it has a size capable of being taken by sucking in as through a drinking straw. Specifically, the shaped solid for oral ingestion having a size of about 0.01 to 30 mm can be stored. In particular, the powder means that, in a formulation size test, a total amount can pass a No. 18 sieve (850 μm), and 5% or less of the total amount cannot pass a No. 30 sieve (500 μm). The fine granule means that it satisfies the requirement of the powder and 10% or less of the total amount can pass a No. 200 sieve (75 μm). The granule means that, in a formulation size test, a total amount can pass a No. 10 sieve (1700 μm), 5% or less of the total amount cannot pass a No. 12 sieve (1400 μm), and 15% or less of the total amount can pass a No. 42 sieve (355 μm). The capsule means that it has a major axis of 10 to 25 mm (No. 0 to No. 5), and the tablet means that it has a diameter of 3 to 30 mm. In the conventional method, the shaped solid for oral ingestion alone was inserted into the mouth, and then water taken from a cup or the like to swallow the same. Therefore, if many capsules or tablets must be taken as a single unit dose, pain or a difficulty was caused. It is preferable to apply the present invention to the formulations under such circumstances.

The shaped solid for oral ingestion is not limited, so long as it is a solid capable of being orally taken, and has a shape, but is for example, medicines, supplements, foods, beverages, or the like. Specifically, it means a shaped solid to be swallowed without manducating while the shape is maintained, that is, to be drunk down without biting. In other words, it means a shaped solid difficult to be swallowed by itself, and thus to be swallowed with water. When the present invention is applied to a large capsule or tablet, a remarkably advantageous effect can be recognized. However, the present invention can be applied to a small granule. Further, when the present invention is applied to a water practically insoluble or insoluble shaped solid, an advantageous effect can be recognized. However, the present invention man be applied to a water soluble or water dispersible shaped solid. Furthermore, when the present invention is applied to a shaped solid which is not destroyed in a short period of time when inserted into the mouth in the conventional method, an advantageous effect can be recognized. The present invention may be applied to an easily-destroyed shaped solid which can substantially maintain its shape for at least 10 seconds after being brought into contact with water. In the present specification, the term “can substantially maintain its shape” in a short period of time after being brought into contact with water means that 80% or more of the shape is maintained after being brought into contact with water.

There may be mentioned as the medicine, for example, powders (including fine granules), granules, capsules, tablets, pills, or the like. Particularly, water practically insoluble or insoluble powders (including fine granules) or granules are, for example, an adsorbent for oral administration or the like.

Any drinking liquid may be used as the liquid for taking the shaped solid for oral ingestion stored in the storage chamber of the unsealed housing body. Most preferably, water is used, but tea, milk, jelly liquid, juice or the like may be used.

The present invention also relates to a container for use in the housing body for deglutition of the shaped solid for oral ingestion. More particularly, the vacant container which does not contain the shaped solid for oral ingestion is supplied to a person taking the shaped solid for oral ingestion. When the person is to take the solid, the person can charge the shaped solid for oral ingestion in the storage chamber, and then suck in and swallow the same.

The container according to the present invention has the same embodiment as that of the present housing body, except that it does not store the shaped solid for oral ingestion therein. Because the container does not store the shaped solid for oral ingestion, it is not necessary to install the unsealable structure part in the opening-side end part. However, it is preferable to install the unsealable structure part in the opening-side end part, to keep the vacant storage chamber clean.

The housing body of the present invention may be used not only by dipping the unsealed housing body into water in a cup, but also by attaching it to a drinking aperture of a jug. In this case, the unsealed housing body of the present invention having a shape attachable to the tip of the drinking aperture of a jug is attached to the tip of the drinking aperture of the jug, and the shaped solid for oral ingestion stored in the unsealed housing body can be swallowed using water in the jug.

### EXAMPLES

The present invention now will be further illustrated by, but is by no means limited to, the following Examples.

### Example 1

(1) Production of a Container for a Net-Type Housing Body

A container for the net-type housing body 10 as shown in FIG. 1 was produced. Specifically, a shielding-side end part B of a polypropylene straw (inner diameter=9 mm; length=210 mm) as a tubular envelope 1 was equipped with a nylon filter (aperture=133 μm; shape=square of 30 mm×30 mm) as a net 32. The nylon filter was fixed by covering with a polypropylene tube (inner diameter=11 mm; length=20 mm) as a locking ring 8 to obtain a net-type housing body 10. In Examples 1 to 4, the resulting container for the net-type housing body 10 was used.

(2) Filling of Capsules

The polypropylene straw, that is, the container produced in the above item (1) for the net-type housing body 10 was vertically set up while the shielding-side end part B was directed downward. Ten capsules in a dry state were charged thereto from the opening part 22 at the top of the opening-side end part A. The capsule used was prepared by filling 250 mg of granular crystalline cellulose (particle size=300 μm to 500 μm; Celphere CP305; Asahi Kasei Chemicals Corp.) into No. 1 capsule of Sionogi Qualicaps, using a capsule filling machine (LZ64, Zanasi). The opening-side end part A was heat-sealed by a heat-sealer (Fuji Impulse) so that the capsules could not be discharged. The No. 1 capsule used had a major axis diameter of 19.7 mm, and a minor axis diameter of 6.7 mm.

### Example 2

The procedures described in Example 1 (1) and (2) were repeated except that 2 g of granular crystalline cellulose (Celphere CP305; Asahi Kasei Chemicals Corp.; particle size=300 to 500 μm) was used instead of the capsules.

### Example 3

The procedures described in Example 1 (1) and (2) were repeated except that 2 g of spherical activated carbons (Kureha-A-BAC; Kureha Chemical Industry Co., Ltd.; particle size=850 to 1190 μm) was used instead of the capsules.

### Example 4

The procedures described in Example 1 (1) and (2) were repeated except that five tablets were used instead of the five capsules used in Example 1.

The tablets used were prepared by forming tablets (diameter=7 mm; thickness=3 mm) from a mixture of 99% by weight of lactose and 1% by weight of talc by a rotary tableting machine (HT-9; Hata Tekkojo), and coating the product with a film of hydroxypropylmethylcellulose 2910 (TC-5; Shin-Etsu Chemical Co., Ltd.).

<<Tests for Swallowing Ability>>

### (1) Example 1 and Comparative Example 1

The housing body 10 produced in Example 1 was used as the housing body 10 of the present invention and compared with a case (Comparative Example 1) wherein ten capsules were taken as in the conventional manner without inserting them into the housing body 10 produced in Example 1.

Specifically, an evaluation was made by ten healthy volunteers. That is, an evaluation of ease of taking was made by comparing the case (Comparative Example 1) wherein they held ten capsules in their mouths, and thereafter took the capsules with water, and the case (Example 1) wherein they dipped the housing body 10 produced in Example 1 in water in a glass, and sucked in the capsules together with the water. The volunteers gave points in accordance with the criteria shown in Table 1, and an amount consumed when taking was recorded. The results are shown in Table 2.

TABLE 1

| Easiness of taking | Score |
|---|---|
| Full capsules not taken. | 1 point |
| Full capsules not taken in a single gulp, | 2 points |
| however taken in two to three gulps. | |
| Full capsules taken in a single gulp, | 3 points |
| however with difficulty. | |
| Full capsules taken in a single gulp, | 4 points |
| without any difficulty. | |

TABLE 2

| | Results | | | |
|---|---|---|---|---|
| | Comparative Example 1: | | Example 1: | |
| | Conventional formulation | | Present housing | |
| | (capsules) | | body formulation | |
| | | Amount of | | Amount of |
| Volunteer | | water | | water |
| No. | Score | consumed | Score | consumed |
| 1 | 1 point | 65 mL | 4 points | 16 mL |
| 2 | 2 points | 69 mL | 4 points | 22 mL |
| 3 | 2 points | 95 mL | 4 points | 24 mL |
| 4 | 3 points | 69 mL | 4 points | 28 mL |
| 5 | 2 points | 84 mL | 4 points | 36 mL |
| 6 | 2 points | 63 mL | 4 points | 37 mL |
| 7 | 2 points | 76 mL | 4 points | 40 mL |
| 8 | 1 point | 64 mL | 4 points | 42 mL |
| 9 | 1 point | 63 mL | 4 points | 47 mL |
| 10 | 1 point | 78 mL | 4 points | 54 mL |
| Average | 1.7 point | 73 mL | 4.0 points | 35 mL |
| score | | | | |

As apparent from the results shown in Table 2, the average score was 1.7 and the average amount of water consumed was 73 mL, in the conventional formulation, whereas the average score was 4.0 and the average amount of water consumed was 35 mL when taken from the housing body 10 of the present invention produced in Example 1. That is, the ease of taking drugs, i.e., the swallowing ability, was improved for all of the ten volunteers.

### (2) Example 2 and Comparative Example 2

The housing body 10 produced in Example 2 was used as the housing body 10 of the present invention and compared with a case (Comparative Example 2) wherein 2 g of the fine granules used in Example 2 was wrapped in a sachet (4 cm×7 cm) and taken together with water in the conventional manner.

Specifically, an evaluation was made by ten healthy volunteers. That is, an evaluation of ease of taking was made by comparing the conventional method (Comparative Example 2) wherein they took the fine granules wrapped with the sachet together with water, and the case (Example 2) wherein they dipped the housing body 10 produced in Example 2 in water in a glass, and sucked in the granules together with the water. The volunteers gave points in accordance with the criteria shown in Table 3, and an amount consumed when taking was recorded. The results are shown in Table 4.

TABLE 3

| Easiness of taking | Score |
|---|---|
| Full fine granules not taken. | 1 point |
| Full fine granules taken in a single gulp, | 2 points |
| however with uncomfortable. | |
| Full fine granules taken in a single gulp, | 3 points |
| without uncomfortable. | |

TABLE 4

| | Results | | | |
|---|---|---|---|---|
| | Comparative Example 2: | | Example 2: | |
| | Conventional formulation | | Present housing | |
| | (fine granules) | | body formulation | |
| | | Amount of | | Amount of |
| Volunteer | | water | | water |
| No. | Score | consumed | Score | consumed |
| 1 | 2 points | 70 mL | 3 points | 36 mL |
| 2 | 3 points | 48 mL | 3 points | 38 mL |
| 3 | 2 points | 40 mL | 2 points | 37 mL |
| 4 | 3 points | 63 mL | 3 points | 40 mL |
| 5 | 2 points | 80 mL | 3 points | 45 mL |
| 6 | 2 points | 64 mL | 3 points | 47 mL |
| 7 | 2 points | 71 mL | 2 points | 53 mL |
| 8 | 2 points | 30 mL | 3 points | 55 mL |
| 9 | 2 points | 45 mL | 3 points | 58 mL |
| 10 | 2 points | 50 mL | 3 points | 62 mL |
| Average | 2.2 points | 56 mL | 2.8 points | 47 mL |
| score | | | | |

As apparent from the results shown in Table 4, the average score was 2.2 and the average amount of water consumed was 56 mL, in the conventional taking method, whereas the average score was 2.8 and the average amount of water consumed was 47 mL when taken from the housing body 10 of the present invention produced in Example 2. That is, the average score was improved, the amount of consumed was reduced, and the ease of taking drugs, i.e., the swallowing ability, was improved.

### (3) Example 3 and Comparative Example 3

The housing body 10 produced in Example 3 was used as the housing body 10 of the present invention and compared with a case (Comparative Example 3) wherein 2 g of the granules used in Example 3 was wrapped in a sachet (4 cm×7 cm) and taken together with water in the conventional manner.

Specifically, an evaluation was made by ten healthy volunteers. That is, an evaluation of ease of taking was made by comparing the conventional method (Comparative Example 3) wherein they took the granules wrapped with the sachet together with water, and the case (Example 3) wherein they dipped the housing body 10 produced in Example 3 in water in glass, and sucked in the granules together with the water. The volunteers gave points in accordance with the criteria shown in Table 3, and an amount consumed when taking was recorded. The results are shown in Table 5.

TABLE 5

| | Results | | | |
|---|---|---|---|---|
| | Comparative Example 3: | | Example 3: | |
| | Conventional formulation | | Present housing | |
| | (granules) | | body formulation | |
| | | Amount of | | Amount of |
| Volunteer | | water | | water |
| No. | Score | consumed | Score | consumed |
| 1 | 3 points | 69 mL | 2 points | 34 mL |
| 2 | 2 points | 47 mL | 3 points | 36 mL |
| 3 | 3 points | 38 mL | 2 points | 37 mL |
| 4 | 2 points | 63 mL | 3 points | 39 mL |
| 5 | 2 points | 76 mL | 3 points | 43 mL |
| 6 | 3 points | 65 mL | 3 points | 47 mL |
| 7 | 3 points | 69 mL | 3 points | 50 mL |
| 8 | 2 points | 35 mL | 3 points | 53 mL |
| 9 | 2 points | 45 mL | 3 points | 55 mL |
| 10 | 2 points | 46 mL | 3 points | 56 mL |
| Average | 2.4 points | 55 mL | 2.8 points | 45 mL |
| score | | | | |

As apparent from the results shown in Table 5, the average score was 2.4 and the average amount of water consumed was 55 mL, in the conventional taking method, whereas the average score was 2.8 and the average amount of water consumed was 45 mL when taken from the housing body 10 of the present invention produced in Example 3. That is, the average score was improved, the amount of water consumed was reduced, and the ease of taking drugs, i.e., the swallowing ability, was improved.

### (4) Example 4 and Comparative Example 4

The housing body 10 produced in Example 4 was used as the housing body 10 of the present invention and compared with a case (Comparative Example 4) wherein five capsules and five tablets used in Example 4 were taken together with water as in the conventional manner.

Specifically, an evaluation was made by ten healthy volunteers. That is, an evaluation of ease of taking was made by comparing the case (Comparative Example 4) wherein they took five capsules and five tablets together with water according to the conventional method, and the case (Example 4) wherein they dipped the housing body 10 produced in Example 4 in water in a glass, and sucked in the capsules and tablets together with the water. The volunteers gave points in accordance with the criteria shown in Table 1, and an amount consumed when taking was recorded. The results are shown in Table 6.

TABLE 6

| | Results | | | |
|---|---|---|---|---|
| | Comparative Example 4: | | Example 4: | |
| | Conventional formulation | | Present housing | |
| | (Capsules + Tablets) | | body formulation | |
| | | Amount of | | Amount of |
| Volunteer | | water | | water |
| No. | Score | consumed | Score | consumed |
| 1 | 2 points | 60 mL | 4 points | 22 mL |
| 2 | 2 points | 65 mL | 4 points | 24 mL |
| 3 | 2 points | 87 mL | 4 points | 25 mL |
| 4 | 2 points | 70 mL | 4 points | 28 mL |
| 5 | 2 points | 81 mL | 4 points | 38 mL |
| 6 | 2 points | 60 mL | 4 points | 38 mL |
| 7 | 2 points | 72 mL | 4 points | 42 mL |
| 8 | 2 points | 64 mL | 4 points | 45 mL |
| 9 | 2 points | 60 mL | 4 points | 48 mL |
| 10 | 2 points | 72 mL | 4 points | 55 mL |
| Average | 2.0 points | 69 mL | 4.0 points | 37 mL |
| score | | | | |

As apparent from the results shown in Table 6, the average score was 2.0 and the average amount of water consumed was 69 mL, in the conventional taking method, whereas the average score was 4.0 and the average amount of water consumed was 37 mL when taken from the housing body 10 of the present invention produced in Example 4. That is, the score in the present invention was full marks for all of the ten volunteers, the amount of water consumed was reduced to half the amount, and the ease of taking drugs, i.e., the swallowing ability, was improved.

### INDUSTRIAL APPLICABILITY

The housing body for deglutition according to the present invention stores the shaped solid for oral ingestion, such as capsules or tablets in a drinking straw-like cylindrical envelope. Thus, as water or the like is drunk through a drinking straw, the shaped solid can be drunk down (i.e., swallowed) together with water after the tip of the cylindrical envelope is dipped in water in a glass. Therefore, a relatively large capsule or tablet can be very easily drunk, i.e., swallowed.

Although the present invention has been described with reference to specific embodiments, various changes and modifications obvious to those skilled in the art are possible without departing from the scope of the appended claims.

### BRIEF DESCRIPTION OF THE DRAWINGS

FIG. 1 is a partial sectional view illustrating a net-type housing body of the present invention wherein capsules are stored in the tubular envelope and the opening-side end part is unsealably heat-sealed, while a part thereof is omitted.

FIG. 2 is a sectional view illustrating the shielding-side end part in the net-type housing body of the present invention wherein the shielding-side end part is crowned with a detachable protective cap.

FIG. 3 is a sectional view illustrating the shielding-side end part in the net-type housing body of the present invention wherein the shielding-side end part is sealed with a sealable protective cap.

FIG. 4 a partial sectional view of the net-type housing body of the present invention dipped in water in a glass.

FIG. 5 is a sectional view of the shielding-side end part in the net-type housing body in accordance with another embodiment of the present invention.

FIG. 6 is a sectional view of the shielding-side end part in the net-type housing body in accordance with still another embodiment of the present invention.

FIG. 7 is a sectional view of the shielding-side end part in the net-type housing body of the present invention wherein the shielding-side end part is sealed with a detachable protective cap.

FIG. 8 is a sectional view of the shielding-side end part in the net-type housing body of the present invention wherein the shielding-side end part is sealed with an unsealable structure part.

FIG. 9 is a perspective view of the shielding-side end part in the narrow passage-type housing body of the present invention.

FIG. 10 is a sectional view of the shielding-side end part in the narrow passage-type housing body of FIG. 9.

FIG. 11 is a perspective view of the shielding-side end part in the narrow passage-type housing body according to another embodiment of the present invention.

FIG. 12 is a sectional view of the shielding-side end part in the narrow passage-type housing body of FIG. 11.

FIG. 13 is a perspective view of the shielding-side end part in the narrow passage-type housing body according to still another embodiment of the present invention.

FIG. 14 is a sectional view of the shielding-side end part in the narrow passage-type housing body according to still another embodiment of the present invention.

FIG. 15 is a sectional view of the shielding-side end part in the narrow passage-type housing body according to still another embodiment of the present invention.

FIG. 16 is a perspective view of a strip-type shielding-side end part in the narrow passage-type housing body according to still another embodiment of the present invention.

FIG. 17 is a perspective view of a tubular storage end part having strips, for producing the housing body of FIG. 16.

FIG. 18 is a perspective view of the opened shielding-side end part of the narrow passage-type housing body according to another embodiment of the present invention.

FIG. 19 is a perspective view of the shielding-side end part of the present invention before unsealing the shielding-side end part of the opened narrow passage-type housing body of FIG. 18.

FIG. 20 is a perspective view of the shielding-side end part in the narrow passage-type housing body according to still another embodiment of the present invention.

FIG. 21 is a sectional view of the shielding-side end part with a backflow preventing valve, in the net-type housing body of the present invention.

FIG. 22 is a partial sectional view illustrating the state that the unsealed housing body having a curved portion according to the present invention is dipped in water in a glass.

FIG. 23 is a partial sectional view illustrating the state that the unsealed housing body having an accordion-folding portion according to the present invention is dipped in water in a glass.

FIG. 24 is a partial sectional view of the opening-side end part in the unsealed housing body with a means for preventing capsules from falling out, according to the present invention.

FIG. 25 is a partial perspective view of the opening-side end part in the unsealed housing body with a means for preventing capsules from falling out, according to another embodiment of the present invention.

FIG. 26 is a partial perspective view of the opening-side end part in the unsealed housing body with a means for preventing capsules from falling out, according to still another embodiment of the present invention.

FIG. 27 is schematic partial sectional view of the opening-side end part in the unsealed housing body with a means of FIG. 26 for preventing capsules from falling out, according to the present invention.

FIG. 28 is a partial perspective view of the opening-side end part in the unsealed housing body with a means for preventing capsules from falling out, according to another embodiment of the present invention.

FIG. 29 is a schematic partial sectional view of the opening-side end part in the unsealed housing body with a means of FIG. 28 for preventing capsules from falling out, according to the present invention.

FIG. 30 is a partial sectional view illustrating the state that the unsealed housing body according to another embodiment of the present invention is dipped in water in a glass.

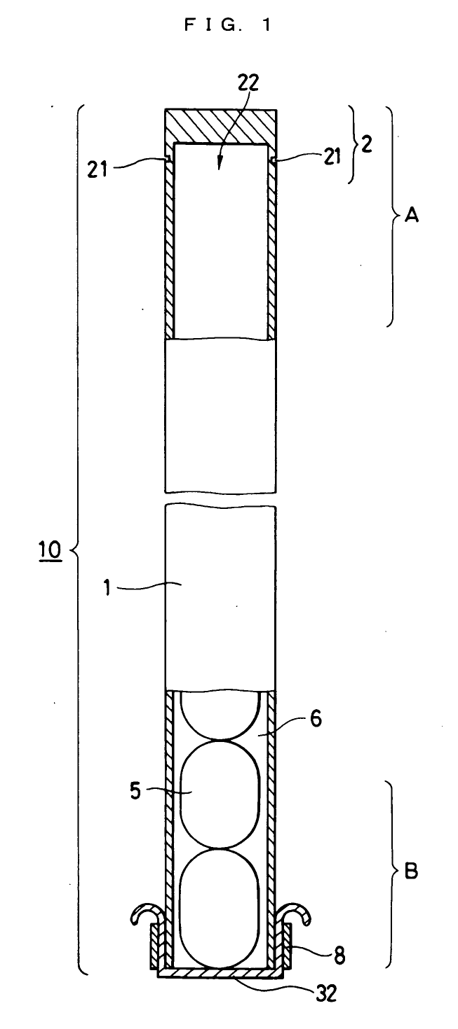

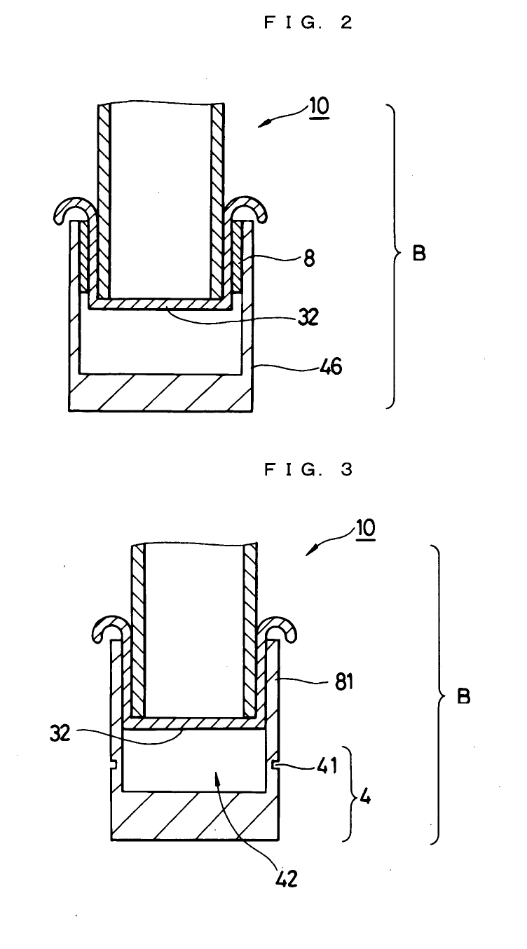

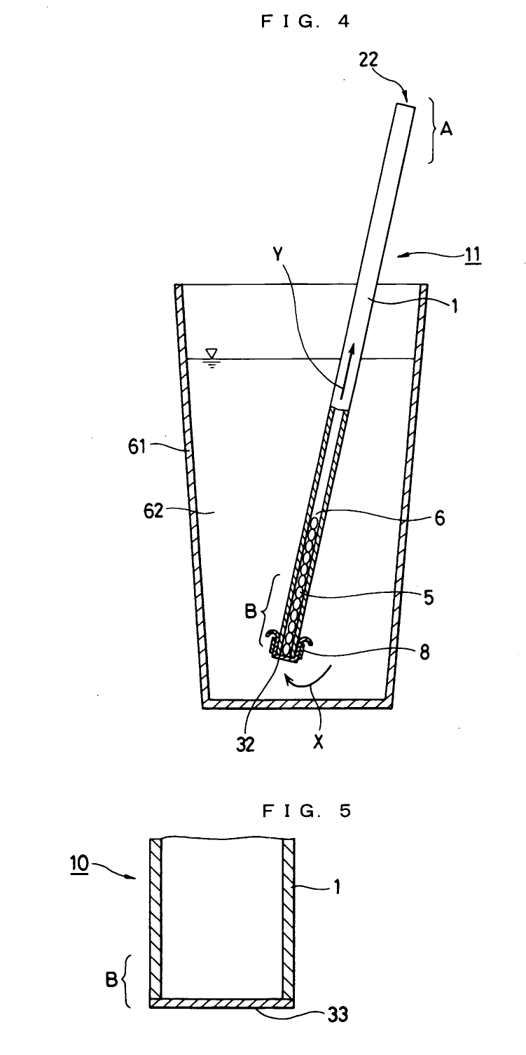

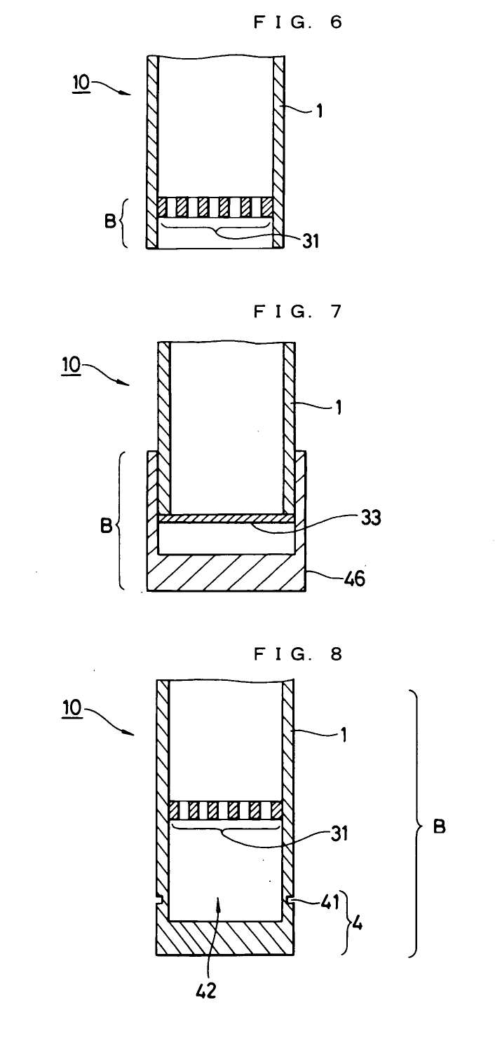

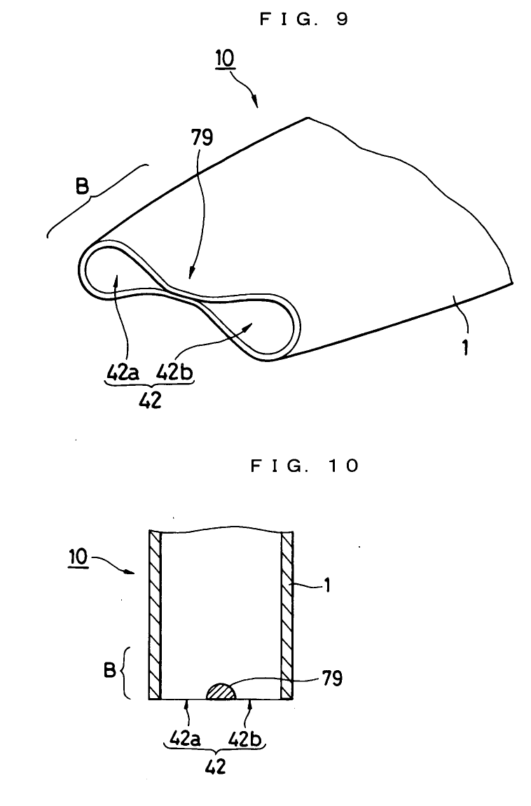

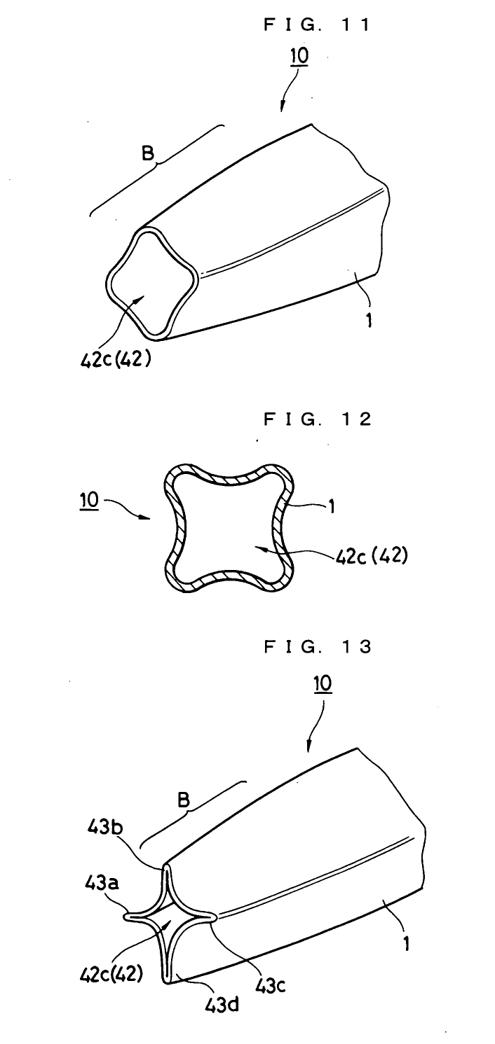

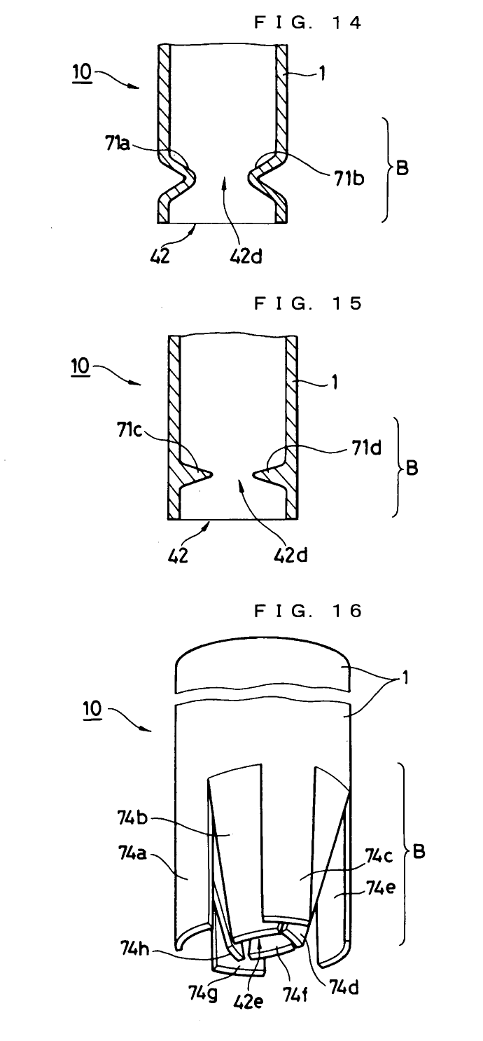

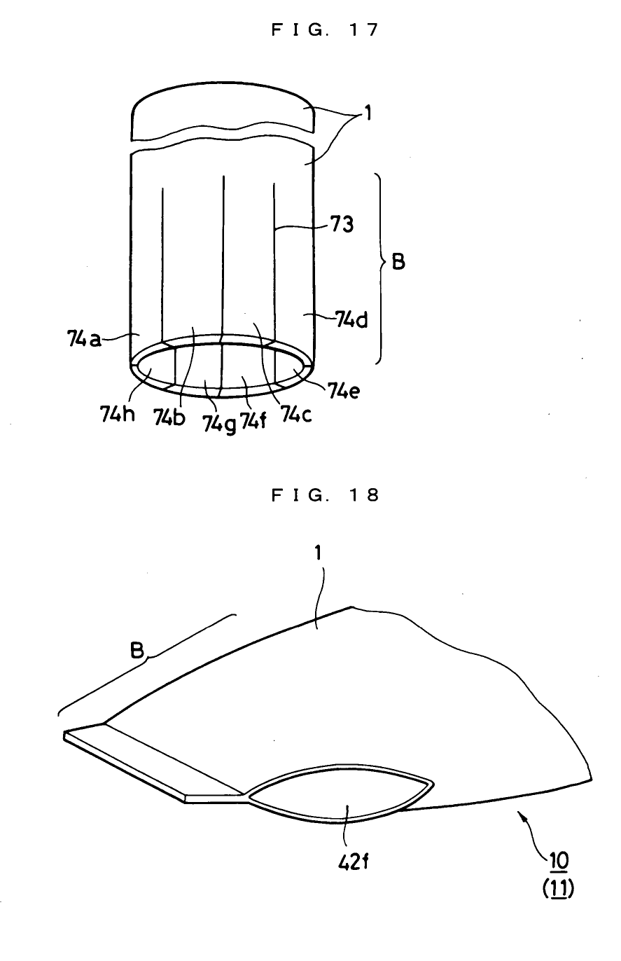

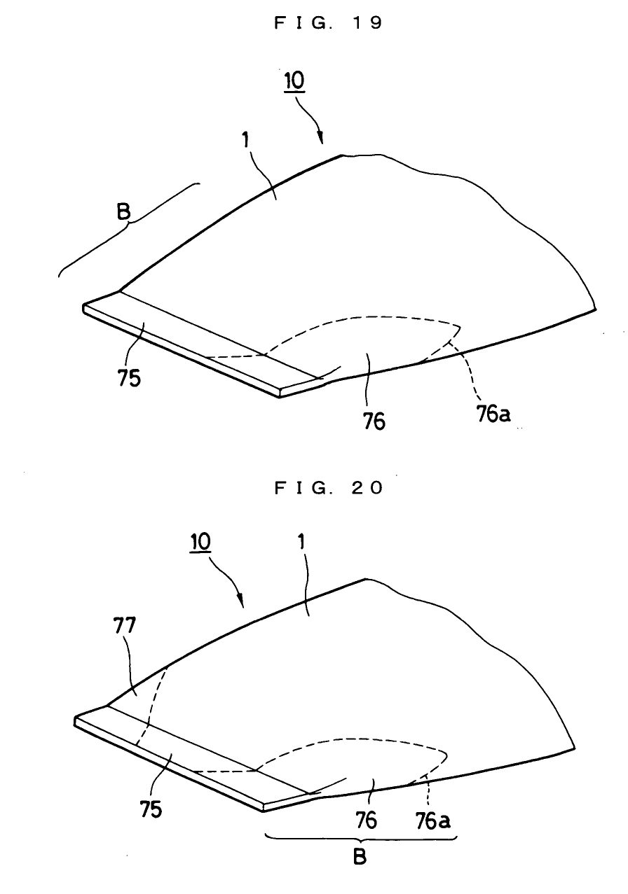

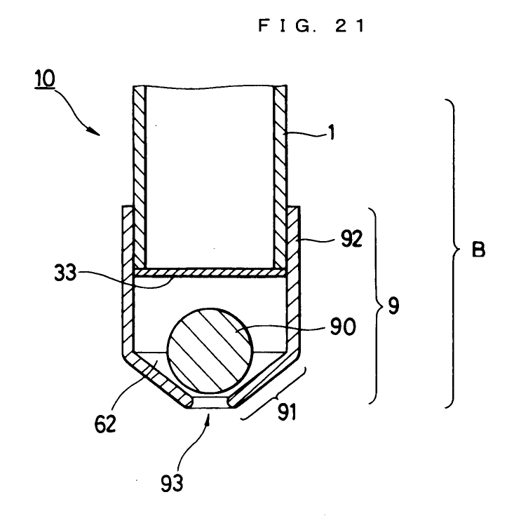

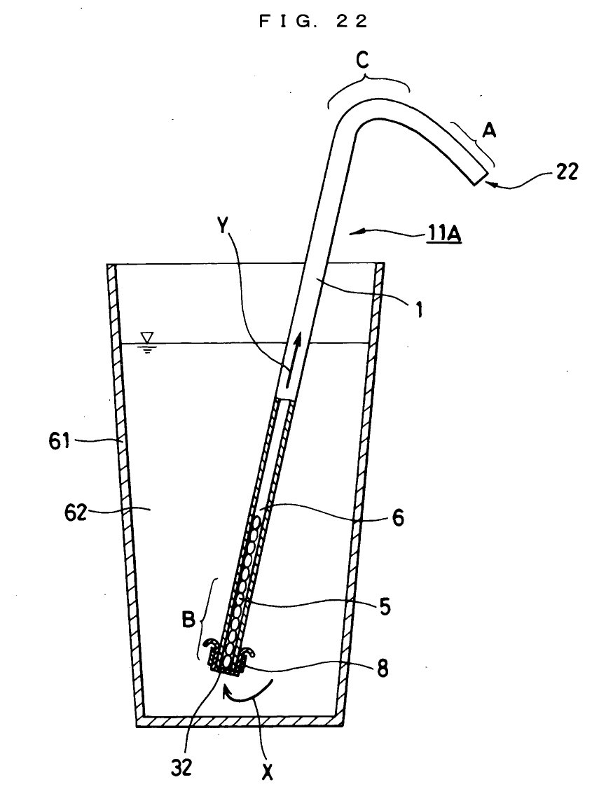

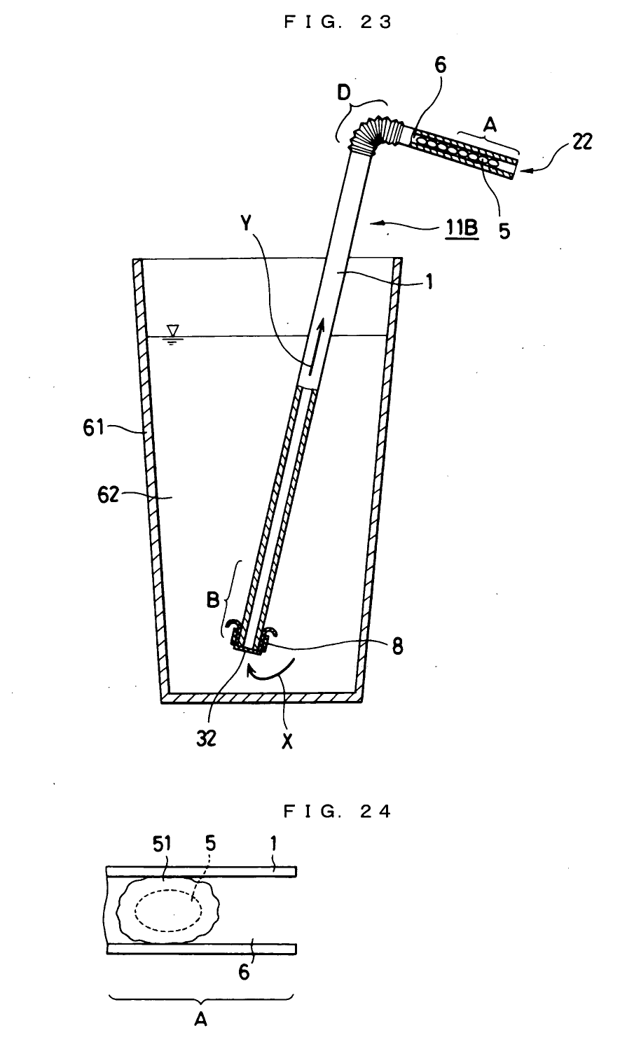

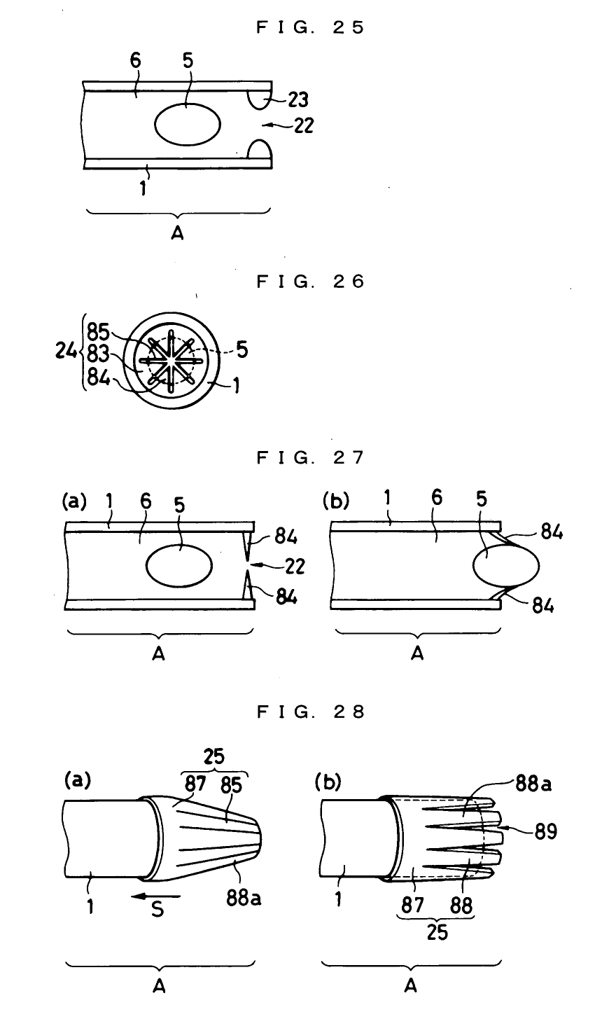

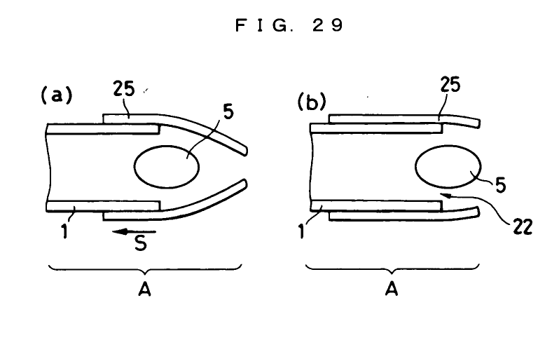

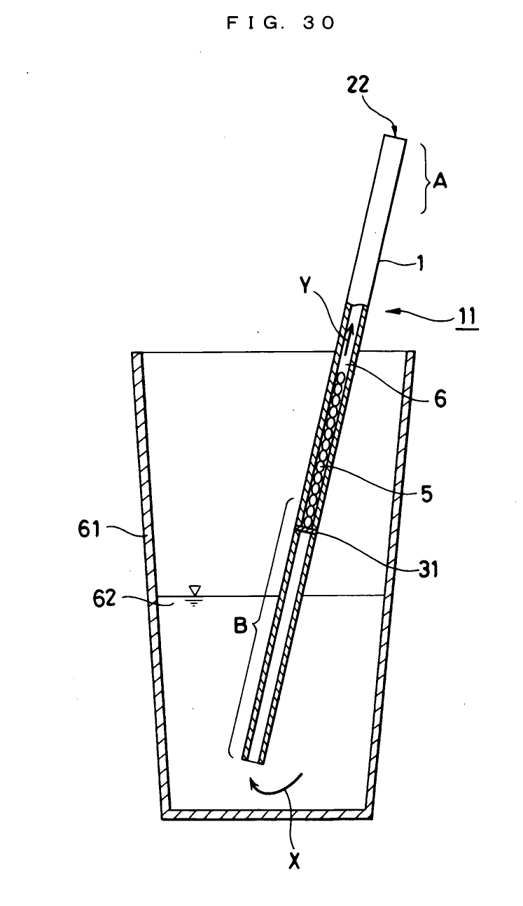

### EXPLANATION OF NUMERICAL REFERENCES

1 . . . tubular envelope; 2 . . . unsealable structure part;
4 . . . unsealable structure part;
5 . . . shaped solid for oral ingestion; 6 . . . storing chamber;
8 . . . locking ring; 9 . . . backflow preventing cap;
10 . . . housing body; 11, 11A, 11B . . . unsealed housing body;
21, 41, 76a . . . groove; 22, 42, 93 . . . opening part;
23 . . . water-soluble protrusions; 24 . . . extruding valve;
25 . . . sliding openable/closable cap; 31, 32, 33 . . . net;
42a, 42b, 42d . . . narrow passage; 42c, 42f . . . narrow opening;
43a, 43b, 43c, 43d . . . joined point; 46 . . . protective cap;
51 . . . water-soluble sheet material; 61 . . . vessel; 62 . . . liquid;
71a, 71b, 71c, 71d . . . protrusion; 73 . . . cutting line;
74a, 74b, 74c, 74d, 74e, 74f, 74g, 74h . . . strip;
75 . . . heat-sealed portion; 76, 77 . . . removable portion;
79 . . . central part;
81 . . . locking cap; 83 . . . ring support;
84 . . . V-shaped folding strip;
85 . . . radial gap; 87 . . . cylindrical support;
88 . . . conical openable/closable part;
88a . . . flexible trapezoidal strip; 89 . . . cutting line;
90 . . . ball; 91 . . . conical jacket; 92 . . . ring joined part;
A . . . opening-side end part; B . . . shielding-side end part; C . . . curved portion; D . . . accordion-folding portion.

1. A housing body for deglutition of a shaped solid for oral ingestion which is stored therein, comprising
(1) a tubular water-resistant envelope;
(2) an opening-side end part at one end of said envelope, having an unsealable structure part, and capable of forming an opening part by removing said unsealable structure part;
(3) a shielding-side end part at the other end of said envelope, having a means for preventing said shaped solid for oral ingestion from release, said means allowing liquid to pass therethrough but not allowing said shaped solid for oral ingestion to pass therethrough; and
(4) a storage chamber storing said shaped solid for oral ingestion between said opening-side end part and said shielding-side end part.

2. The housing body according to claim 1, wherein said means for preventing said shaped solid for oral ingestion from release is a net entirely covering a cross-sectional area of said tubular envelope, or a narrow passage portion formed by a single or plural protrusions projecting towards an inner space of said tubular envelope.

3. The housing body according to claim 1, further comprising a backflow preventing means located adjacent to said means for preventing said shaped solid for oral ingestion from release, and on the opposite side to said storage chamber.

4. The housing body according to claim 1, wherein an unsealable structure part is also placed at an outermost edge of said shielding-side end part.

5. The housing body according to claim 1, wherein said tubular water-resistant envelope is of a linear, spiral, or curved form from one end to the other end, or said tubular water-resistant envelope is a linear envelope having a spiral or curved portion, or a portion transformable to a curved form, as a part thereof.

6. The housing body according to claim 1, wherein said shaped solid for oral ingestion is a molding capable of being swallowed down together with water without mastication.

7. The housing body according to claim 1, wherein said shaped solid for oral ingestion is a powder (including fine granule), granule, capsule, tablet, or pill.

8. The housing body according to claim 7, wherein said powder (including fine granule) or granule is an adsorbent for oral administration.

9. The housing body according to claim 7, wherein a major axis diameter of said tablet is 3 to 30 mm.

10. The housing body according to claim 7, wherein a major axis size of said capsule is 10 to 25 mm (No. 0 to 5).

11. The housing body according to claim 1, wherein said shaped solid for oral ingestion is a shaped solid capable of substantially maintaining a shape thereof for at least 10 seconds after being brought into contact with water.

12. The housing body according to claim 1, wherein a single unit dose of said shaped solid for oral ingestion is stored.

13. A method for swallowing a shaped solid for oral ingestion, characterized in that
(1) an unsealable structure part installed at an opening-side end part of the housing body according to claim 1, and an unsealable structure part optionally installed at a shielding-side end part thereof are removed,
(2) said shielding-side end part of said housing body is immersed in liquid in a vessel while said shaped solid for oral ingestion is stored in said storage chamber in said tubular envelope, and
(3) an opening part formed by removing said unsealable structure part installed at said opening-side end part is held between the lips so that said shaped solid for oral ingestion is sucked in together with said liquid in said vessel.

14. A container for use in a housing body for deglutition of a shaped solid for oral ingestion, comprising
(1) a tubular water-resistant envelope;
(2) an opening-side end part at one end of said envelope, having an unsealable structure part, and capable of forming an opening part by removing said unsealable structure part;
(3) a shielding-side end part at the other end of said envelope, having a means for preventing said shaped solid for oral ingestion from release, said means allowing liquid to pass therethrough but not allowing said shaped solid for oral ingestion to pass therethrough; and
(4) a storage chamber capable of storing said shaped solid for oral ingestion between said opening-side end part and said shielding-side end part.

15. A method for swallowing a shaped solid for oral ingestion, characterized in that
(1) an unsealable structure part installed at an opening-side end part of the container according to claim 14 is removed,
(2) said shaped solid for oral ingestion is introduced in the storing chamber of the tubular envelope from an opening part formed by removing said unsealable structure part installed at said opening-side end part,
(3) the shielding-side end part is immersed in liquid in a vessel while said shaped solid for oral ingestion is stored in the storage chamber, and
(4) an opening part at said opening-side end part is held between the lips so that said shaped solid for oral ingestion is sucked in together with liquid in said vessel.

16. A container for use in a housing body for deglutition of a shaped solid for oral ingestion, comprising
(1) a tubular water-resistant envelope;
(2) an opening-side end part at one end of said envelope,
(3) a shielding-side end part at the other end of said envelope, having a means for preventing said shaped solid for oral ingestion from release, said means allowing liquid to pass therethrough but not allowing said shaped solid for oral ingestion to pass therethrough; and
(4) a storage chamber capable of storing said shaped solid for oral ingestion between said opening-side end part and said shielding-side end part.
